(19) United States
(12) Patent Application Publication
(10) Pub. No.: US 2023/0056171 A1
(43) Pub. Date: Feb. 23, 2023

(54) STERILIZING DEVICE AND METHOD FOR STERILIZING AN OUTER FACE OF A RECEPTACLE

(71) Applicant: Syntegon Technology GmbH, Waiblinger (DE)

(72) Inventors: Ulrich Krauß, Ilshofen (DE); Matthias Angelmaier, Crailsheim (DE); Andreas Bühler, Neresheim (DE)

(21) Appl. No.: 17/797,307

(22) PCT Filed: Feb. 10, 2021
(86) PCT No.: PCT/EP2021/053130

§ 371 Date: Aug. 3, 2022

(30) Foreign Application Priority Data
Feb. 10, 2020 (DE) 10 2020 103 327.3

(51) Int. Cl. A61L 2/20; A61L 2/10

(52) U.S. Cl. CPC A61L 2/208; A61L 2/10; A61L 2202/11; A61L 2202/23

(57) ABSTRACT

The present invention relates to a sterilizing device (10) and to a method for sterilizing an outer face of a receptacle (22).

### BACKGROUND

The present invention relates to a sterilizing device and to a method for sterilizing an outer face of a receptacle.

When filling packaging with pharmaceutical products, it is necessary for the primary packaging that is in contact with the pharmaceutical products to not be contaminated with living microorganisms (including bacteria, fungi, etc.). Otherwise, the pharmaceutical products will not keep and will become unusable within a short period of time due to contamination or pose a risk to patients.

In order to ensure sterility when filling containers, the containers, e.g., vials, syringes, etc., are first cleaned with water and then sterilized.

Known forms of sterilization include, for example, the use of heat, radioactive radiation, toxic gases, and the like. These cleaning and sterilization processes are relatively complex. They are often not carried out by those filling packaging with pharmaceutical products themselves, but are instead increasingly outsourced to the packaging manufacturers. The primary packaging is cleaned by the packaging manufacturer, packed appropriately, and then sterilized completely by means of a toxic gas.

The packing can be carried out, for example, using a plastics carrier plate (also referred to as a nest), a plastics trough (also referred to as a tub) having a lidding foil adhesively bonded thereto, and at least one or two protective bags that form outer packaging. The protective bags together with the tubs contained therein are packed in cardboard boxes, which can be transported and stored on pallets. Sterilization takes place with the entire pallet, and therefore the outer packaging and foils used are designed to be gas-permeable. The protective bags and lidding foils are made of a material that is gas-permeable, but impermeable to bacterial germs. Corresponding materials are known from the prior art. One form of container packaging of this kind is standardized according to ISO 11040-7.

In the following, a unit consisting of a plastics trough or tub (gas-impermeable) and a lidding foil (gas-permeable) adhesively bonded thereto is also referred to as a receptacle, or constitutes an example of a receptacle. Any protective bags which are typically designed to be gas-permeable are also referred to as outer packaging.

When such containers or receptacles packed as described are brought into a sterile space, for example, care must be taken to ensure that no living microorganisms are carried over from the outside of the outer packaging into the sterile region.

Various methods are known from practice for making the unpacked receptacle or tub outer face sterile. In systems having a high output, for example, the tub is irradiated with electron beams. This is disadvantageous in that X-rays are produced in the process, and so lead shields are necessary for protecting the machine operators. Methods using UV irradiation or other high-energy (light) radiation are also known, but these may not result in a 6-log depletion of germs (depletion of the germs is to 0.001 per thousand of the original amount). Particularly in uneven regions, due to shadowing, germs can only be killed to an insufficient extent.

Gaseous hydrogen peroxide (H2O2), which also has a germ-killing effect over a certain exposure time, can also be used. The hydrogen peroxide in the gas phase (gas/mist/aerosol mixture) may come into contact with the primary packaging, inside the receptacle, via the gas-permeable outer packaging and lidding foil and may remain as a residue at a certain residual concentration (in the ppm range). This can have negative effects on very sensitive medicines to be filled. There are also applications that involve other germ-killing gases, but they have similar disadvantages.

Plasma sterilization is also known from practice, but is associated with high costs. It is also possible to transfer the receptacle/tub directly from the outer packaging (protective bag) into the sterile space. This is based on the assumption that the inside of the outer packaging (protective bag) is likely enough to already be sterile. However, the aseptic safety of this practice is sometimes rejected as being too uncertain.

It has also been proposed to remove the receptacle/tub from the bag and then adhesively bond a gas-impermeable foil, for example an aluminum foil, to the lidding foil. The receptacle covered in this way should then be decontaminated with gaseous H2O2. In the sterile region, the lidding foil and the adhesively bonded foil are then removed together so that no germs can be exposed. This is disadvantageous in that there is the additional process of adhesively bonding the foil. There is also the risk that germs will remain on the edges of the two foils, since these bonded points are only insufficiently accessible to the gas used for sterilization.

### SUMMARY

The present inventions (sterilizing device and method) now provide an option for effective and safe decontamination/sterilization, with combined decontamination/sterilization being carried out. The receptacle is sterilized in the region of the gas-impermeable receptacle body primarily by exposure to hydrogen peroxide (H2O2) in the gas phase, while the region of the gas-permeable lidding foil is sterilized in a radiation-based manner and there is only little or negligible contact with hydrogen peroxide. As a result, a sufficient reduction in germs can be achieved and hydrogen peroxide is effectively prevented from being able to come into contact with the primary packaging in the receptacle via the gas-permeable lidding foil.

The sterilizing device according to the invention for sterilizing an outer face of a receptacle accordingly comprises a transfer lock and optionally a removal region. The sterilizing device is used to sterilize receptacles to be sterilized. The receptacles have a gas-permeable lidding foil and a gas-impermeable receptacle body. The lidding foil is also designed to be impermeable to germs. The receptacle body is usually designed in the manner of a trough. The receptacle body delimits a receiving space which is accessible via a removal opening in the receptacle body. The removal opening is closed by the lidding foil. In an initial state, the receptacle (in particular in the form of a tub with a nest arranged therein with primary packaging for medical purposes arranged in the nest) is initially arranged in outer packaging designed, for example, as a bag or protective bag. In other words, the sterilizing device is used to sterilize or decontaminate the outer surface of the receptacle when it is removed from the outer packaging.

The removal region that may optionally be provided comprises a circulation device. The circulation device is designed and arranged to flush a cleaned gas around the receptacle while it is being removed from the outer packaging in the removal region. From the removal region, the receptacle removed from the outer packaging is transferred to a transfer lock.

The transfer lock comprises a cover unit. The cover unit can be arranged relative to the receptacle such that the cover unit covers the region of the receptacle formed by the lidding foil. For this purpose, the cover unit can be designed to be movable, but it can also be provided that the cover unit is arranged so as to be immovable and the receptacle in question can be placed under the cover unit in an automated manner, for example.

The cover unit comprises a radiation source. The receptacle or its lidding foil can be irradiated with electromagnetic radiation by means of the radiation source. The radiation source can be a UV radiation source. This provides for irradiation that is as harmless as possible for operators, but reliable. It is also possible to use other high-energy light radiation of specific wavelengths. Suitable wavelengths are to be selected preferably such that they correspond as closely as possible to the absorption wavelengths of the microorganisms, viruses, etc., to be killed.

The transfer lock also comprises a decontamination unit. An atmosphere containing H2O2 (hydrogen peroxide) can be generated in the transfer lock by means of the decontamination unit. The decontamination unit can be designed to introduce hydrogen peroxide into the atmosphere in the region of the transfer lock, in a gaseous state or as a mist. For this purpose, the decontamination unit can comprise an evaporation or nebulization device.

When the sterilizing device is in operation, the receptacle is removed from its outer packaging in the removal region. The receptacle is then transferred to the transfer lock. The transfer lock can be designed such that the space in which the receptacle together with the cover unit is now arranged is sealed off or can be sealed off in as gas-tight a manner as possible, which reduces the use of hydrogen peroxide. The receptacle is placed in the transfer lock in the intended position with respect to the cover unit. In this intended position, the cover unit covers the region of the lidding foil, which will be discussed in detail later. The decontamination unit is then activated and generates an atmosphere containing hydrogen peroxide in the transfer lock. At the same time or with a time delay (in particular shortly thereafter), the region of the lidding foil of the receptacle is irradiated (for example with UV radiation) by means of the radiation source provided in the cover unit. The region of the receptacle on the receptacle body side is therefore sterilized by means of hydrogen peroxide and the region of the lidding foil is exposed to UV radiation and thereby sterilized. Since the cover unit is arranged spatially very close to the lidding foil in the intended position, the hydrogen-containing atmosphere can enter the region of the lidding foil and pass through the lidding foil only very slowly. The cover unit can also contact the lidding foil in the intended position. If the decontamination process is terminated after a period of time sufficient for decontaminating the outer face of the receptacle, that time will not have been sufficient for allowing hydrogen peroxide to pass from the surrounding atmosphere into the interior of the receptacle. The transfer lock can then be flushed with a hydrogen-peroxide-free gas (e.g., cleaned air) and the receptacle can be removed from the transfer lock.

The cover unit comprises a flat cover side which comprises a cover plate (for example made of glass, in particular UV-permeable glass) that is permeable to the radiation from the radiation source. This cover plate can be arranged in parallel with the lidding foil, which usually extends in a planar manner, at the smallest possible distance therefrom (but also in contact with the lidding foil). Contact between the cover plate and the lidding foil is typically avoided. However, a gap between the cover plate and the lidding foil is formed with the smallest possible gap width in order to suppress or slow down the entering of the hydrogen peroxide in the environment into the gap as much as possible.

The circulation device in the removal region can be designed to form a directed, low-turbulence gas flow in order to allow the flow to be flushed around the receptacle as effectively as possible.

Correspondingly, the decontamination unit in the transfer lock can also be designed to form a directed, low-turbulence gas flow, which, on the one hand, causes the flow to be flushed around the receptacle body or its outer face effectively. On the other hand, the laminar flow of the gas keeps the amount of gas containing hydrogen peroxide being introduced into the gap between the cover unit and the lidding foil as low as possible.

The aspects and possible developments of the sterilizing device just described in connection with the sterilizing device can also be part of the method described below. Conversely, the aspects of the method described below and its developments can also be advantageous developments of the sterilizing device according to the invention.

The method according to the invention for sterilizing an outer face of a receptacle relates to receptacles as described at the outset above in connection with the sterilizing device.

Consequently, such a receptacle has the gas-permeable lidding foil and the gas-impermeable receptacle body. The receptacle body delimits the receiving space, which is in turn accessible via the removal opening in the receptacle body. The removal opening is closed by the lidding foil.

The method according to the invention now provides for a cover unit to be placed in the region of the lidding foil such that inflow, or the possibility of the inflow, of gas between the cover unit and the lidding foil is reduced, and for subsequent sterilization of the receptacle body outer face by means of a H2O2-containing gas with simultaneous sterilization of the lidding foil by means of electromagnetic radiation, in particular UV radiation.

At the beginning of the method, the receptacle can be initially arranged, in an initial state, in outer packaging designed in particular as a bag. In particular, the method comprises the steps described below, it also being possible for the steps to each individually represent a development of the method.

In step 1, the receptacle is introduced into the removal region, in which a cleaned gas is circulated around the receptacle. In this step, the receptacle is still in the outer packaging.

In step 2, the receptacle is removed from the outer packaging while the cleaned gas is circulating therearound. This removal can take place, for example, automatically by means of a removal apparatus, which is typically also arranged in the removal region. The removal apparatus can also comprise an automated opening device that opens the outer packaging.

In step 3, the receptacle is introduced into a transfer lock. For this purpose, a transport unit can be provided, which is designed to automatically transport the receptacle from the removal region into the transfer lock.

In step 4, the receptacle is arranged in the region of a cover unit such that the cover unit covers the region of the receptacle formed by the lidding foil. This can take place directly after the transfer to the transfer lock, for example by automatically transporting the receptacle to under the cover unit. The cover unit can then, for example, still be lowered toward the receptacle (or the receptacle is raised toward the cover unit). Optionally, the region of the transfer lock around the receptacle and the cover unit can still be closed in a gas-tight manner.

In step 5, the transfer lock is flooded with a H2O2-containing atmosphere with simultaneous irradiation or subsequent irradiation of the lidding foil of the receptacle by means of an electromagnetic radiation source arranged in the cover unit (typically UV radiation, typically with intensity components in the range between 245 nm and 300 nm, in particular with intensity maxima in this range, in particular with intensity components in the range from 250 nm to 280 nm, in particular with intensity maxima in this range). Light with a high energy density of wavelengths in the range of 200-350 nm is generally provided for irradiating the lidding foil, so that said light reacts as well as possible with the DNA of the microorganisms.

In step 6, the transfer lock is flushed with H2O2-free gas and the receptacle is removed from the transfer lock.

As a result, the region of the receptacle that is sensitive to hydrogen peroxide, i.e., the region of the lidding foil, is largely protected from being in contact with the hydrogen peroxide in the atmosphere in the transfer lock and is only sterilized with the electromagnetic radiation, which, as already outlined above, is mainly in the form of UV radiation.

As already mentioned, it is advantageous for the cover unit to comprise a flat cover side which comprises a cover plate that is permeable to the radiation from the radiation source, for the lidding foil of the receptacle to be designed and arranged so as to extend in a planar manner, and for the lidding foil and the cover plate to be aligned in parallel with one another before the H2O2-containing atmosphere is generated in the transfer lock. Typically, the smallest possible gap (distance) is left between the cover plate and the lidding foil (but contact is also possible), so that, while the lidding foil is not contacted and possibly damaged, the gap is so small that the inflowing hydrogen peroxide is only very delayed, for example enters the gap by diffusion processes which have a speed that is lower by some orders of magnitude than the convective transport processes that prevail in the remaining space of the transfer lock. As a result, the lidding foil is only in very little contact with hydrogen peroxide and said hydrogen peroxide cannot penetrate the interior of the receptacle via the lidding foil. The UV radiation cleans or sterilizes the region of the lidding foil to a sufficient extent, however.

As already mentioned, the cover plate and the lidding foil are kept at a distance from one another, but in close proximity to one another, while the H2O2-containing atmosphere exists in the transfer lock. In particular, they are kept at a distance (gap width) from one another of less than 2 cm, in particular less than 1.5 cm, in particular less than 1 cm, in particular less than 0.5 cm. However, contact between the cover plate and the lidding foil is also possible. This distance can be constant or decrease over time (e.g., from an initial value which corresponds to the values just mentioned), for example by moving the cover unit toward the receptacle, or vice versa. As a result, the gas located between the cover unit and the lidding foil can be pushed outward, which leads to the introduction of hydrogen peroxide being slowed down further. The cover unit and the receptacle are typically moved toward one another so slowly that the flow that forms between the cover unit and the receptacle is laminar.

The circulation in step 1 can take place with a directed, laminar flow. It can also be provided that, in step 5, a directed, laminar flow of H2O2-containing gas is circulated around the receptacle (good contact of the receptacle body with hydrogen peroxide and at the same time a low amount of hydrogen peroxide introduced into the gap between the receptacle and the cover unit).

The receptacle body can be designed in the manner of a trough and a holder comprising containers/primary packaging (e.g., vials or syringes) provided for medical substances and accommodated in the holder can be arranged in the receiving space of the receptacle that is closed by the lidding foil.

Typically, the receptacle body can be designed as what is referred to as a tub. Its upper side is usually open or only closed by means of the lidding foil. The entire area of the lidding foil is typically covered by the cover unit. The cover unit can also project laterally beyond the receptacle or lidding foil. However, overlapping of parts of the receptacle that are formed only by the receptacle body is typically kept as low as possible, since these regions can be effectively sterilized with hydrogen peroxide. Conversely, the radiation-emitting region of the cover unit extends in particular over the entire area of the lidding foil. In other words, it is provided in particular that the cover plate, which is permeable to the radiation from the radiation source, has at least the extent of the lidding foil or protrudes laterally therebeyond. Correspondingly, it can be provided in particular that a plurality of radiation sources is provided or a planar radiation emission of the radiation sources is provided. It is also possible for the cover plate to leave the outermost edge of the sealing seam of the lidding foil free in order to achieve reliable sterilization of the edge region using H2O2.

It can be provided that the gas for producing the atmosphere containing hydrogen peroxide in the transfer lock is fed to a catalyst after it has flowed out of the transfer lock in order to break down the hydrogen peroxide content.

It can be provided in particular that gas containing hydrogen peroxide flows past the receptacle in a laminar flow from sides of the cover unit and is carried away from the receptacle via a return air duct on a side of the receptacle arranged opposite the cover unit.

### BRIEF DESCRIPTION OF THE DRAWINGS

Further features, possible applications and advantages of the inventions result from the following description of embodiments of the inventions, which are explained with reference to the drawings, where the features may be essential to the inventions, both in isolation and in different combinations, without being explicitly mentioned again. In the drawings:

FIG. 1 shows a device according to the invention, which carries out a method according to the invention;

FIG. 2 shows a portion from FIG. 1;

FIG. 3 is a schematic process sequence.

### DETAILED DESCRIPTION

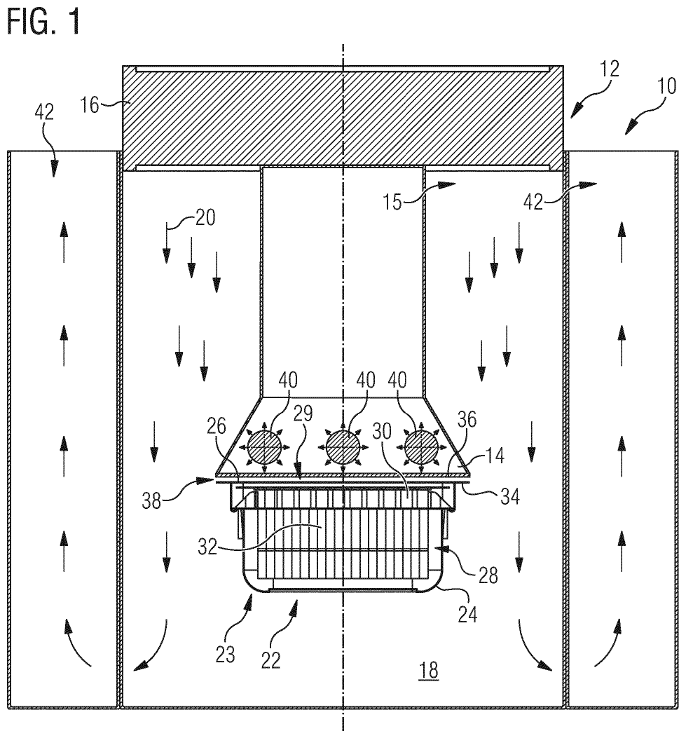

FIG. 1 shows a sterilizing device 10 and its transfer lock 12 in detail. A removal region of the sterilizing device 10 is not shown in the figure.

The transfer lock 12 comprises a cover unit 14, a decontamination device 15 and a filter unit 16. The transfer lock 12 is designed having a space 18 that can be sealed in a gas-tight manner. Gas 20 containing hydrogen peroxide, the direction of flow of which is represented by arrows, can be introduced into the space 18 via the decontamination device 15.

A receptacle 22 to be sterilized and having an outer face 23 is arranged in the space 18 below the cover unit 14. The receptacle 22 comprises a receptacle body 24 and a lidding foil 26 which together form the outer face 23. The receptacle body 24 is designed in the manner of a trough and comprises a receiving space 28. The receiving space 28 opens into a removal opening 29 (pointing upward in the present case), which is closed by the lidding foil 26. In the present example, the receiving space 28 is consequently delimited by the receptacle body 24 and the lidding foil 26, in the present case completely (without further delimiting elements).

A nest 30 is arranged inside the receiving space 28 and comprises primary packaging 32 designed as syringes. The lidding foil 26 is connected to the receptacle body 24 at a laterally protruding edge 34. The lidding foil 26 is planar and even. The receptacle body 24 is impermeable to both germs and gases, in particular H2O2 in the gas phase (gaseous or as a mist/aerosol). The lidding foil 26 is impermeable to germs. The lidding foil 26 is permeable to gases, in particular H2O2 in the gas phase (gaseous or as a mist/aerosol).

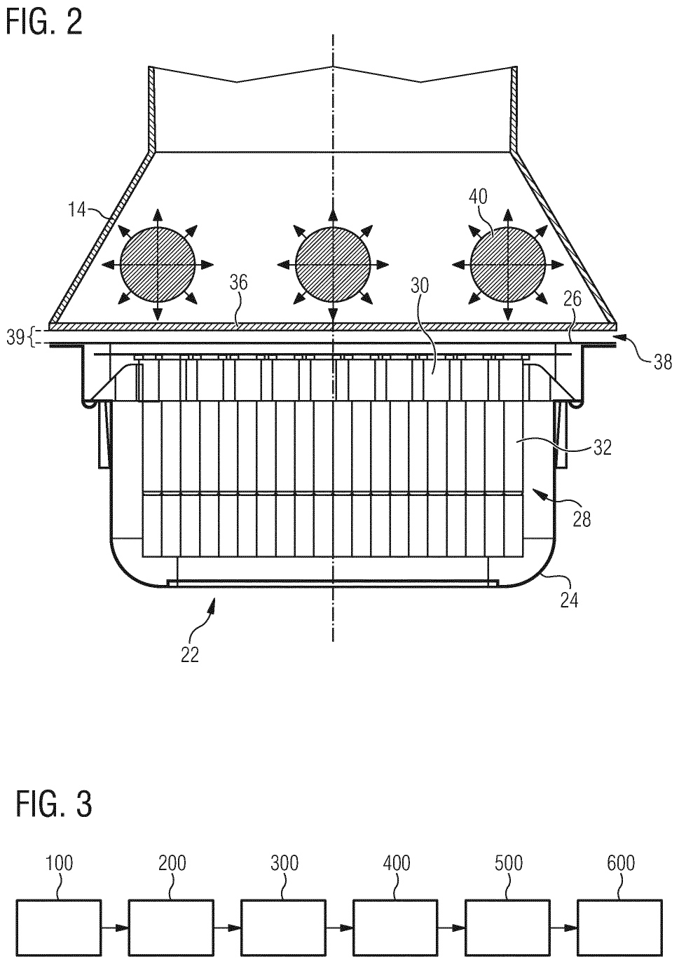

The region around the cover unit 14 and the receptacle 22 is shown enlarged in FIG. 2.

In the state shown in FIGS. 1 and 2, the receptacle 22 is arranged such that the lidding foil is arranged in parallel with a cover plate 36 of the cover unit 14. A gap 38 is formed between the lidding foil and the cover plate, which in the present case is formed having a gap width 39 of 0.5 cm.

The cover unit 14 comprises a plurality of radiation sources 40 which are designed to emit UV radiation and can each emit UV radiation in the direction of the lidding foil 26 through the cover plate 36. For this purpose, the cover plate 36 is designed to be UV-permeable. This is illustrated by corresponding arrows emanating from the radiation sources 40.

The method according to the invention for sterilizing the receptacle 22 is illustrated schematically in FIG. 3 using the example of the operation of the sterilizing device 10.

In a first step 100, the receptacle 22 in outer packaging is introduced into a removal region of the sterilizing device 10 and a cleaned gas, for example cleaned air, circulates therearound with a directed, laminar flow.

In a second step 200, the receptacle 22 is removed from the outer packaging while the cleaned gas is circulating therearound.

In a third step 300, the receptacle 22 is introduced into the transfer lock 12, as is shown in FIG. 1, for example.

In a fourth step 400, the receptacle 22 is arranged in the region of the cover unit 14 such that the cover unit 14 covers the region of the receptacle 22 formed by the lidding foil. For this purpose, the cover unit 14 can be held stationary and the receptacle 22 can be moved, or vice versa. It is also possible for receptacle 22 and cover unit 14 to be moved in this step.

In a fifth step 500, the transfer lock 12 is flooded with a H2O2-containing atmosphere. At the same time (directly before or after is also possible), the lidding foil 26 of the receptacle 22 is irradiated by means of the electromagnetic radiation source 40 arranged in the cover unit 14, which is designed as a UV radiation source in the example from FIG. 1.

In order to flood the transfer lock with the H2O2-containing atmosphere, the gas 20 flows past the receptacle 22 in a laminar flow from the side of the cover unit 14. On a side of the receptacle 22 arranged opposite the cover unit 14, the gas 20 is carried away from the receptacle 22 via a return air duct 42 or a plurality of return air ducts 42, which are arranged to the side of the cover unit 14. The gas 20 can then be cleaned, for which purpose, for example, catalytic decomposition of the H2O2 in the gas can be used.

In a sixth step 600, the transfer lock is flushed with H2O2-free gas and the receptacle 22 is removed from the transfer lock 12.

1. A sterilizing device (10) for sterilizing an outer face (23) of a receptacle (22), the device (10) comprising a transfer lock (12), wherein the receptacle (22) to be sterilized has a gas-permeable lidding foil (26) and a gas-impermeable receptacle body (24) which delimits a receiving space (28) which is accessible via a removal opening (29) in the receptacle body (24), wherein the removal opening (29) is closed by the lidding foil (26), wherein the transfer lock (12) comprises a cover unit (14) having a radiation source (40), which can be arranged relative to the receptacle such that the cover unit (14) covers a region of the receptacle (22) formed by the lidding foil (22) and can irradiate the lidding foil (22) with electromagnetic radiation, wherein the transfer lock (12) also comprises a decontamination unit (15), by which a H2O2-containing atmosphere can be generated in the transfer lock (12), wherein the cover unit (14) comprises a flat cover side which comprises a cover plate (36) that is permeable to the radiation from the radiation source (40).

2. (canceled)

3. The sterilizing device (10) according to claim 1, wherein the radiation source (40) is a UV radiation source (40).

4. The sterilizing device (10) according to claim 1, wherein the decontamination unit (15) in the transfer lock (12) is designed to form a directed, low-turbulence gas flow.

5. The sterilizing device (10) according to claim 1, wherein the decontamination unit (15) for generating the H2O2-containing atmosphere H2O2 in the transfer lock (12) is designed to introduce H2O2 in a gaseous state or as a mist.

6. A method for sterilizing an outer face (23) of a receptacle (22), wherein the receptacle (22) has a gas-permeable lidding foil (26) and a gas-impermeable receptacle body (24) which delimits a receiving space (28) which is accessible via a removal opening (29) in the receptacle body (24), and wherein the removal opening (29) is closed by the lidding foil (26), wherein the method comprises:
placing a cover unit (14) in the region of the lidding foil (26) such that a possibility of an inflow of gas (20) between the cover unit (14) and the lidding foil (26) is reduced, and subsequently sterilizing the outer face of the receptacle body (24) by H2O2-containing gas (20) while simultaneously sterilizing the lidding foil (26) by electromagnetic radiation.

7. The method according to claim 6, wherein, in an initial state, the receptacle (22) is initially arranged in outer packaging wherein the method comprises the following steps:
step 1: introducing the receptacle (22) in the outer packaging into a removal region in which a cleaned gas circulates around the receptacle (22);
step 2: removing the receptacle (22) from the outer packaging with the cleaned gas circulating therearound;
step 3: introducing the receptacle (22) into a transfer lock (12);
step 4: arranging the receptacle (22) in the region of the cover unit (14) such that the cover unit (14) completely or at least predominantly covers the region of the receptacle (22) formed by the lidding foil (26);
step 5: flooding the transfer lock (12) with a H2O2-containing atmosphere with simultaneous and/or prior or subsequent irradiation of the lidding foil (26) of the receptacle (22) by an electromagnetic radiation source (40) arranged in the cover unit (14);
step 6: flushing the transfer lock (12) with H2O2-free gas and removing the receptacle (22) from the transfer lock (12).

8. The method according to claim 6, wherein the cover unit (14) comprises a flat cover side which comprises a cover plate (36) that is permeable to the radiation from a radiation source (40), and the lidding foil (26) of the receptacle (22) is designed and arranged so as to extend in a planar manner, and the lidding foil (26) and the cover plate (36) are aligned in parallel with one another before the H2O2-containing atmosphere is generated in the transfer lock (12).

9. The method according to claim 6, wherein while the H2O2-containing atmosphere exists in the transfer lock (12), the cover plate (36) and the lidding foil (26) are in contact with one another or at a distance from one another.

10. The method according to claim 7, wherein the circulation in step 1 takes place with a directed, laminar flow, and/or in that, in step 5, a directed, laminar flow of H2O2-containing gas (20) circulates around the receptacle (22).

11. The method according to claim 6, wherein the receptacle body (24) is a trough, and a holder (30) comprising primary packaging (32) provided for medical substances and accommodated in the holder (30) is arranged in the receiving space (28) that is closed by the lidding foil (26).

12. The sterilizing device (10) according to claim 1, wherein the device further includes a removal region, and wherein, in an initial state, the receptacle (22) is initially arranged in outer packaging, wherein the removal region comprises a circulation device which is designed and arranged to flush a cleaned gas around the receptacle (22) while it is being removed from the outer packaging in the removal region.

13. The sterilizing device (10) according to claim 12, wherein the circulation device in the removal region is designed to form a directed, low-turbulence gas flow.

14. The sterilizing device (10) according to claim 1, wherein the electromagnetic radiation is high-energy light radiation generated by the radiation source (40)

15. The sterilizing device (10) according to claim 14, wherein the high-energy light radiation is UV radiation.

16. The method according to claim 6, wherein the electromagnetic radiation is UV radiation

17. The method according to claim 9, wherein the cover plate (36) and the lidding foil (26) are at a distance (39) from one another, that is constant or decreases over time, of less than 2 cm.

18. The method according to claim 9, wherein the distance (39) is less than 1.5 cm.

19. The method according to claim 18, wherein the distance (39) is less than 1 cm.

20. The method according to claim 18, wherein the distance (39) is less than 0.5 cm.
